(19) United States
(12) Patent Application Publication
(10) Pub. No.: US 2008/0069791 A1
(43) Pub. Date: Mar. 20, 2008

(54) Means for stimulation and activation of hair growth by il-15

(75) Inventor: Stefan Beissert, Muenster (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Universitaetsklinikum Muenster, Muenster (DE)

(21) Appl. No.: 10/584,766

(22) PCT Filed: Dec. 7, 2004
(86) PCT No.: PCT/EP04/13907

§ 371 Date: Mar. 15, 2007

(30) Foreign Application Priority Data
Dec. 29, 2003 (EP) 03029899.6

(51) Int. Cl. A61K 8/64

(52) U.S. Cl. 424/70.14; 514/2; 514/171; 514/559; 514/389; 514/284; 514/456

(57) ABSTRACT

The present invention relates to the use of IL-15 polynucleotides, polypeptides or compounds which bind to an antibody which specifically recognizes the IL-15 polypeptide or which specifically bind to an IL-15 receptor alpha chain for the preparation of a composition for stimulating hair growth or for treating, preventing and/or ameliorating hair loss. Moreover the present invention encompasses a transgenic non-human animal comprising an IL-15 polynucleotide. The present invention also relates to a method for stimulating hair growth in a non-human animal and to methods for manufacturing animal hair. Finally, the present invention relates to a method of treating, preventing and/or ameliorating a subject which suffers from hair loss.

The present invention relates to the use of IL-1 5 polynucleotides, polypeptides or compounds which bind to an antibody which specifically recognizes the IL-15 polypeptide or which specifically bind to an IL-15 receptor alpha chain for the preparation of a composition for stimulating hair growth or for treating, preventing and/or ameliorating hair loss. Moreover the present invention encompasses a transgenic non-human animal comprising an IL-15 polynucleotide. The present invention also relates to a method for stimulating hair growth in a non-human animal and to methods for manufacturing animal hair. Finally, the present invention relates to a method of treating, preventing and/or ameliorating a subject which suffers from hair loss.

Hair loss is a severe problem in humans from a medical and cosmetic point of view. Hair loss has diverse causes in humans, including inherent or acquired disease conditions and such conditions which are the result of a medical treatment such as chemotherapy or which are caused by environmental influences. In humans by far the most common cause of hair disorders are defects in hair growth control like alopecia areata or androgenic alopecia. Besides humans, hair loss or slow hair growth is a problem of various animals, particular those which are used for the manufacture of hair, such as sheep, horse, lama or camel. The hair is usually obtained from those animals by shaving. After the animal has been shaved it takes a certain time window until hair regrowth is induced and the animal can be shaved again. Said time window is the important rate limiting step in the manufacture of hair by using animals.

Hair growth is governed by the hair follicles which reside in the skin. The hair follicle is a complex cylindrical structure, consisting of different layers of epithelial cells. The outer compartment is the root sheath. This compartment is connected to the epidermis and here cell proliferation takes place (29). Hair follicles are highly dynamic, they remodel themselves repeatedly throughout life, in a cycle of growth (anagen), regression (catagen) and rest (telogen). In the catagen phase proliferation takes place and in the lower part of the hair follicle massive apoptosis can be observed (30). Several cytokines, among them fNF-x, IL-1-B and IFN-y, have been suggested to play a role in controlling and promoting apoptosis in hair bulb keratinocytes during catagen (33). IL-15 was shown to interfere with cyclophosphamide-induced apoptosis in hair follicle keratinocytes (34). IL-15 is a pleiotropic cytokine that is important for immune cell homeostasis as well as peripheral immune finctions (1-4). Numerous in vitro and in vivo studies have demonstrated a critical role for IL-15 in the development and survival of NK cell lineage (5,6). Other studies in mice have shown that IL-15 is a selective growth factor for memory CD8+rather than CD4+T cells as well as for certain subsets of intraepithelial lymphocytes (7-10). Accordingly, IL-15-/- and IL-15Ra-l- mice are lymphopenic, and specifically lack NK cells, NKT cells, intestinally derived subsets of intraepithelial lymphocytes as well as activated CD8+T cells (5,11). Ubiquitous transgenic overexpression of IL-15 under the control of a MHC class I promoter led to the initial expansion of NK and memory phenotype CD8+T cells and then to the development of a fatal leukemia, resulting in premature death of these mutant mice. Therefore, it is difficult to investigate the long-term in vivo effects of IL-15 (6). Furthermore, IL-15 is involved in the anabolic pathway of muscle cells, acts as a growth factor for mast cells, and as a general inhibitor of apoptosis in T cells, B cells, and fibroblasts (12-15). These studies are consistent with the broad expression of IL-15 in multiple cell types and tissues. Accordingly, several reports show abundant IL-15 MRNA transcripts in many tissues and cell types (3,16). However, IL-15 expression is tightly controlled at the levels of transcription, translation, and intracellular transport (17-19). In particular, the IL-15 protein is posttranscriptionally regulated by several controlling elements, e.g. 12 AUGs in the 5′ UTR, that impede translation, two insufficient signal peptides, and a negative regulator at the C-terminus of the precursor protein. Thus, investigations on the in vivo fuiction of IL-15 are greatly impaired. Within the skin the primary cellular source of IL-15 are keratinocytes. Keratinocytes express IL-15 mRNA but produce the IL- 15 protein only upon stimulation such as with ultraviolet radiation or wounding, and in psoriatic lesions (15,20,21). IL-15 transcripts have also been detected in freshly isolated human Langerhans cells (LC). IL-15 was even reported to be involved in the in vitro generation of human LC from monocytes. IL-15 is a 14-15 kDa protein expressed at the mRNA level in numerous tissues and in a broad range of cell types, including activated monocytes, dendritic cells, osteoclasts and fibroblasts (1,3). The heterotrimeric IL-15 receptor (IL-15R) consists of the IL-2R ′3-chain and y-chain, together with a unique oc-chain (IL-1 5Rc) which is responsible for high affinity binding to IL-15. Whereas IL-2Ra is primarily expressed on activated T cells, IL-15Rx mRNA has been identified in various tissues and cells. Like IL-2 the IL- 15Rccy complex signals through JAK1I/3 and STAT3/5 pathways (3,28). IL-15 has already been described to be essential for the proliferation and maintenance of CD8+memory T cells and acts, at high dose, as a pan T cell and mast cell growth factor (2,26,27).

However, effective means for promoting hair growth and for treating, preventing and/or ameliorating the diseases referred to hereinabove are still not obtainable but nevertheless highly desirable.

Thus, the technical problem underlying the present invention must be seen as the provision of means for effectively promoting hair growth and for treating, preventing and/or ameliorating hair loss caused by or accompanied with diseases. The technical problem is solved by the embodiments characterized in the claims.

Accordingly, the present invention relates to the use of
(i) a polynucleotide comprising
   (a) a nucleic acid sequence as shown in SEQ ID NO: 1 or 3,
   (b) a nucleic acid sequence encoding an amino acid sequence as shown in SEQ ID NO: 2 or 4,
   (c) a nucleic acid sequence encoding an amino acid sequence as shown in SEQ ID NO: 2 or 4 having a modified signal peptide, a modified N-terminus and/or a modified C-terminus, or
   (d) a nucleic acid sequence which hybridises under stringent conditions to any one of (a) to (c);
(ii) a polypeptide encoded by the nucleic acid as defined in any one of (a) to (d); or
(iii) a compound which binds to an antibody which specifically recognizes the polypeptide defined in (ii) or which specifically binds to an IL-1 5 receptor alpha chain for the preparation of a composition for stimulating hair growth.

The term “polynucleotide” relates to polynucleotides which encode a polypeptide having a biological or antigenic activity of interleukin 15 (IL-15). The structure of various IL-15 polypeptides has been described in the art and representative IL-15 polypeptides are shown in SEQ ID NO: 2 (human IL-15, Accession No. BC018149; gi34783292) and SEQ ID NO: 4 (mouse IL-15, Accession No. BC023698; gi23271448). Several biological functions of IL-15 are also reported in the art and have been discussed herein before. An essential biological activity of IL-15 is its capability to specifically bind to the IL-15 receptor alpha chain as deposited under (Accession No. BC022705 for mouse IL-15 Ra and Accession No. AY316538 for human IL-15 Ra (2, Lodolce, Immunity 1998, 9: 669 - 676)). Other well characterized biological activities include its capability to stimulate NK-/NKT cells and memory T-cells (Flamand, J. Clin. Invest, 1996, 97: 1373 - 81; Kv, Science 2000, 288: 675-678) and its proliferative effect on lymphoid or mesenchymal cells as well as the prevention of apoptosis after induction with apoptic substances (14). Preferably, said biological activity is the stimulation of hair growth and keratinocytes as demonstrated in the accompanied Examples. An essential antigenic activity is its capability to be specifically recognized by a specific, i.e. non-cross-reactive, IL-15 antibody as disclosed in Shinozcki, J. Clin. Invest, 2002, 109: 951 - 960. Such an IL-15 antibody can also be obtained by routine methods.

Preferably, the antibody is a monoclonal antibody. These activities can be tested by routine methods well known in the art and described in the above cited references in detail. Most preferably, the polynucleotides of the present invention have a nucleic acid sequence as shown in SEQ ID NO: 1 (human IL-1 5) or SEQ ID NO: 3 (mouse IL-1 5). Preferably, the IL-15 polynucleotides also encompass variant polynucleotides which are capable to hybridise with the polynucleotides shown in SEQ ID NO: 1 or SEQ ID NO: 3 under stringent hybridisation conditions. More preferably, said conditions are disclosed in Ausubel, 2001, Current protocols in molecular biology. Said polynucleotides are most preferably at least 70 %, at least 80 %, at least 85 %, at least 90 %, at least 95 %, at least 96 %, at least 97 %, at least 98 % or at least 99 % identical with SEQ ID NO: 1 or SEQ ID NO: 3.

The variant polynucleotides of the invention may comprise a modified signal peptide or leader sequence, i.e. amino acids 1 to 48 of SEQ ID NO: 2, amino acids 1 to 48 of SEQ ID NO: 4 and amino acids corresponding thereto in polypeptide variants thereof. Modifications meant hereby are those which increase the secretion of IL-15 from a cell. Biological assays for testing whether a modification increases said secretion are well known in the art and are described in (5) and (6). Most preferably, the signal peptide is modified by replacing it with the signal peptide of CD33 polypeptide (Accession No. NM 02 1293). Moreover, the N- or C-terminal amino acids of the mature polypeptide shown in SEQ ID NO: 2 or SEQ ID NO: 4 or amino acids corresponding thereto in the polypeptide variants may be modified as to increase stability of the mature polypeptides. The stability of mature IL-15 polypeptides can be tested by routine techniques (5) and (6). Preferred modifications are inserting a FLAG epitope tag to the N-terminal or C-reminal amino acid of the mature IL-1 5. Instead of a FLAG epitope, an HA or myc epitope can be used. Also encompassed by the polynucleotides referred to in connection with the present invention are biologically active fragments of the polynucleotides shown in SEQ ID NO: 1, SEQ ID NO: 3 or its variants specified before. Said fragments may be obtained by deletion of one or more nucleotides of the respective nucleic acid sequences. The fragments can be generated by standard techniques well known to the person skilled in the art.

The term “polypeptide” as used herein encompasses those polypeptides which are encoded by the polynucleotides specified hereinabove and which have at least one, and preferably all, of the aforementioned antigenic or biological activities.

The term “compound” encompasses all classes of chemical entities which bind to an antibody which specifically recognizes the IL-15 polypeptide of the invention or which specifically bind to an IL-15 receptor alpha chain as mentioned above. Whether a given compound exhibits these properties can be tested by routine techniques including those specified hereinbefore. Preferred classes of chemical entities to be used according to the present invention are antibodies such as monoclonal antibodies, polyclonal antibodies, single chain antibodies, human or humanized antibodies, primatized, chimerized antibodies or fragments thereof. Moreover, encompassed are bispecific antibodies, synthetic antibodies, antibody fragments, such as Fab, Fv or scFv fragments etc., or a chemically modified derivative of any of these. Further compounds encompassed by the use of the invention include peptides, proteins, nucleic acids, antibodies, small organic compounds, ligands, peptidomimetics, PNAs and the like. The compounds to be used according to the present invention preferably act as agonists of IL-1 5. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art or as described. Furthermore, peptide mimetics and/or computer aided design of appropriate drug derivatives and analogues can be used. Appropriate computer programs can be used for the identification of interactive sites of a putative agonist of IL-15 by computer assistant searches for similar structural motifs. Further appropriate computer systems for the computer aided design of protein and peptides are described in the prior art, for example, in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. The results obtained from the above-described computer analysis can be used in combination with the method of the invention for, e.g., optimizing known inhibitors, analogs, antagonists or agonists. Appropriate peptidomimetics and other inhibitors can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of said compounds and the polypeptides of the invention can be used for the design of peptidomimetic drugs. It is very well known how to obtain said compounds, e.g. by chemical or biochemical standard techniques. A suitable assay for identifying a compound to be used according to this invention, preferably, comprises the steps of (a) contacting cells known to be responsive to IL-15, preferably keratinocytes, NK or NKT cells, memory and effector or regulatory T cells or lymphoid or mesenchymal cells, and (b) determining the response of said cells to the administration of the compound whereby a response comparable to the response induced by IL-15 is indicative for a compound according to this invention. Preferred responses to be determined are stimulation of growth in the case of keratinocytes, T cell subsets, NK cells or NKT cells or prevention of programmed cell death upon induced by an apoptotic stimulus in the case of lymphoid or mesenchymal cells. How such assays can be carried out is well known in the art (5, 6, 11, 12, 14, 20, 21). Alternatively, a compound to be used according to this invention can be determined by an assay comprising the steps of (a) contacting a specific IL-15 antibody or the IL-15 receptor alpha chain with IL-15 and a candidate compound and (b) determining competition between IL-15 and said compound for antibody or receptor binding. For determination of the competition it is preferred that either the compound or the IL-15 will be linked to a detectable label, such as a radioisotope or a chromogenic chemical entity. Moreover, a compound according to the present invention can be determined by (a) contacting the IL-15 receptor alpha chain with a candidate compound and (b) determining the activation or response of said receptor whereby a response or activation comparable to the response or activation induced by IL-15 is indicative for a compound to be used according to this invention. How such assays can be carried out is well known in the art (5, 6, 11, 12, 14, 20, 21).

The term “composition” refers to any composition formulated in solid, liquid or gaseous form and, inter alia, may appear as a powder, tablet, solution or an aerosol. Said composition comprises the IL-15 polynucleotide, polypeptide or the compound of the invention optionally together with a suitable diluent, excipients and/or carrier. Suitable diluents and/or carriers depend on the purpose for which the composition is to be used and the other ingredients. The person skilled in the art can determine such suitable diluent, excipients and/or carrier without further ado. Examples of suitable carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These compositions can be administered to a subject at a suitable dose. Administration of the compositions may be effected by different ways, e.g., by topical, subcutaneous, intradermal or intravenous administration. It is particularly preferred that said administration is carried out by delivery to an area where hair growth is to be stimulated. This can be done preferably by subcutaneous or epidermal injection or topical application, e.g. in form of solutions or aerosols or by vehicles such as liposomes for nucleic acids or macromolecular cage molecules such as fullerenes. Moreover, if nucleic acids of the invention are to be administered conventional gene therapy approaches can be used. Gene therapy, which is based on introducing therapeutic genes into cells by ex-vivo or in-vivo techniques is one of the most important applications of gene transfer. Transgenic mice expressing a neutralizing antibody directed against nerve growth factor have been generated using the “neuroantibody” technique; Capsoni, Proc. Natl. Acad. Sci. USA 97 (2000), 6826-6831 and Biocca, Embo J. 9 (1990), 101-108. Suitable vectors, methods or gene-delivering systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813, Isner, Lancet 348 (1996), 370-374; Muhlhauser, Circ. Res. 77 (1995), 1077-1086; Onodua, Blood 91 (1998), 30-36; Verzeletti, Hum. Gene Ther. 9 (1998), 2243-2251; Verma, Nature 389 (1997), 239-242; Anderson, Nature 392 (Supp. 1998), 25-30; Wang, Gene Therapy 4 (1997), 393-400; Wang, Nature Medicine 2 (1996), 714-716; WO 94/29469; WO 97/00957; US 5,580,859; US 5,589,466; US 4,394,448 or Schaper, Current Opinion in Biotechnology 7 (1996), 635-640, and references cited therein. The nucleic acid molecules and vectors of the invention may be designed for direct introduction or for introduction via liposomes, viral vectors (e.g. adenoviral, retroviral), electroporation, ballistic (e.g. gene gun) or other delivery systems into the cell. Additionally, a baculoviral system can be used as eukaryotic expression system for the nucleic acid molecules of the invention. The introduction and gene therapeutic approach should, preferably, lead to the expression of functional IL-1 5 polypeptides of the invention, whereby said expressed polypeptides are particularly useful for stimulation of hair growth in a subject. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg/kg body weight per dose. However, doses below or above this exemplary range are envisioned, especially considering the aforementioned factors. If the regimen is a continuous infusion, it should also be in the range of 1 [ig to 10 mg units per kilogram of body weight per minute. Progress can be monitored by periodic assessment. The compositions of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. The composition of the invention may comprise further agents depending on the intended use of the composition. Said agents may be drugs acting on hair growth including those referred to herein below. The term “stimulating hair growth” refers to a significant induction and/or increase in hair growth on skin, scalp or any surface on which hair growth is desired. The induction or increase can be determined in comparison to skin which has not been treated (control skin). Whether the induction or increase is significant can be determined by statistical tests such as Student's t-test, chi²-test or the U-test according to Mann and Whitney. The stimulation of hair growth is required for treating or ameliorating various medical conditions specified herein in detail. Moreover, the stimulation of hair growth may be important and indicated from a cosmetic point of view. For the manufacture of hair for producing goods such as clothes, blankets, furniture etc. stimulation of hair growth is required even on healthy skins in order to minimize the time windows between shaving of the animals and, thus, to increase the yield of hair. Accordingly, stimulation of hair growth is even required on healthy skin or scalp.

IL-15 is a pleiotropic cytokine, which based on its in vitro activities is important for immune cell homeostasis as well as peripheral immune finctions in vivo. To better understand the in vivo effects, a transgenic (tg) mouse model in which IL-1 5 is overexpressed in the epidermis was generated in the study underlying the present invention. The transgene was expressed in the skin for the following reasons: Ubiquitous transgene overexpression of IL-15 under control of a MHC class I promoter caused the development of a fatal leukemia, resulting in premature death of these tg animals (6). Thus, this model does not allow the investigation of long-term in vivo effects of IL-15. Furthermore, the skin was selected since it has been demonstrated that keratinocytes can function as a natural source of IL-15 and that the secretion of IL-15 by these cells is inducible. Moreover, because of its pleiotropic expression IL-15 is supposed be important for the regulation of numerous cell types. Therefore, it was hypothesized that this cytokine may also drive the stimulation, activation or proliferation of cells of the skin.

Surprisingly, it was show in the study underlying the present invention that keratinocyte derived IL-15 in tg animals is able to enhance hair growth and hair development by stimulating the proliferation and or differentiation of hair follicle root sheath cells. Hair follicles are highly dynamic, they remodle themselves repeatedly throughout life, in a cylce of growth (anagen), regression (catagen) and rest (telogen). In the catagen phase proliferation takes place and in the lower part of the hair follicle massive apoptosis can be observed (30). As mentioned above IL-15 increases the proliferation of various cell types. According to the results of the study underlying this invention IL-15 seems also able to drive proliferation of hair follicle cells. Tg animals that were shaved and depilated also showed de novo development of hair follicles as detected by histological analysis and clinical appearance therefore IL-15 may also affect the proliferation of papilla cells or hair stem cells remaining in the dermis after depilation. These results suggest that IL-15 possibly is also necessary for the initiation of a new hair growth cycle. In addition IL-15 is a general inhibitor of apoptosis in T and B cells as well as fibroblast (12-15). Probably this cytokine could also inhibit apoptosis in basal hair follicle cells and therefore it is conceivable that IL-15 enhances hair growth by stimulating the proliferation of hair follicle cells and by inhibiting apoptosis in the lower part of the hair follicle both leading to a prolonged lifespan and higher activity of each follicle in IL-15 tg mice as we were able to show by shaving and depilating the animals. The signals controlling the hair follicle cycle are still incompletely understood. However, many of the major signalling pathways are involved in the cyclical growth. For example the catagen phase is initiated by expression of transforming growth factor (TGF) ox and C whereas in the anagen phase (growth phase) two other factors are mainly active: fibroblast growth factor (FGF) and signal transducer and activator of transcription factor (STAT3; 29,31,32). As already mentioned IL-15, signals (after binding to its recepror) via the STAT pathway suggesting that this cytokine additionally has a direct effect on the growth phase of hairs because IL-15 interferes in a signal pathway that is important for initiating the growth phase. In humans by far the most common cause of hair disorders are defects in hair growth control like alopecia areata or androgenic alopecia. Because of its proliferative and stimulating capacities it is conceivable to use IL-15 to restimulate and reactivate hair follicles. It also will be possible to prevent hair loss due to chemotherapies. Further, IL-1 5 will be useful to, e.g., enhance merino or angora wool production by applying this cytokine to shaved sheep or rabbit skin. Advantageously, due to the identification of IL- 15 as a key regulator of hair growth in vivo in the study underlying the present invention it has become possible to stimulate hair growth in a controlled and efficient manner. In this context it should be emphasized that IL-15 will not merely prevent apoptosis but also stimulate and promote growth of the cells. The interpretation of the terms and the explanations made hereinabove and below apply mutatis mutandis for all embodiments described herein. In light of the foregoing, the present invention relates to the use of a polynucleotide, polypeptide or compound as defined hereinabove for the preparation of a composition for treating, preventing and/or ameliorating hair loss. The term “treating” as used herein means that all clinical symptoms accompanied with a disease condition are absent after said treatment. The term “preventing” means that the clinical symptoms of a disease become not determinable. The term “ameliorating” means that the symptoms of a disease are significantly reduced. Said treatment, prevention or amelioration shall preferably occur in a significant number of subjects to which the composition has been administered. As discussed above, hair loss is a prominent disease condition in humans. Due to the use of the present invention it has become advantageously possible to prevent hair loss during the early stages of onset and to stimulate new hair growth on shaved skin or after hair was lost after chemotherapy. In a preferred embodiment of the use of the invention said composition further comprises a second hair growth stimulating agent. The term “second hair growth stimulating agent” relates to agents which are also capable of stimulating hair growth significantly. Whether an agent is capable of stimulating hair growth can be determined as specified above. More preferably, said second hair growth stimulating agent is selected from the group consisting of zinc salts of carboxylic acids, saponins, triterpenes, preferably oleanolic acid or ursolic acid, crataegolic acid, celastrol, Asiatic acid, inhibitors of 5-[alpha]-reductase, preferably progesterone, 1,4-methyl-4-azasteroids, preferably 17-[beta]-N,N-diethylcarbamoyl-4-methyl-4-aza-5-[alpha]-androstan-3-one, androgen receptor antagonists, preferably cyproterone acetate, Minoxidil(R), azaelaic acid and derivatives thereof, cyclosporin, triiodothyronine, diazoxide, potassium channel openers, preferably cromakalin, phenytoin, and mixtures thereof, and derivatives of oestrogen, preferably oestradiolvalerate. The structure of theses agents is described in detail in US2003114526 the disclosure of which is hereby incorporated by reference. Moreover as specified above, in another preferred embodiment of the use of the invention, said composition further comprises a pharmaceutically or cosmetically acceptable carrier. Suitable pharmaceutically or cosmetically acceptable carriers are disclosed in detail hereinabove and below. Moreover, such carriers are disclosed in US2003114526 the disclosure of which is hereby incorporated by reference. In a further preferred embodiment of the use of the invention said composition is a pharmaceutical composition. The term “pharmaceutical composition” as used herein comprises the substances of the present invention and optionally one or more pharmaceutically acceptable carrier. The substances of the present invention may be formulated as pharmaceutically acceptable salts. Acceptable salts comprise acetate, methylester, HCl, sulfate, chloride and the like. The pharmaceutical compositions can be conveniently administered by any of the routes conventionally used for drug administration, for instance topically. The substances may be administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like referred to above. Similarly, the carrier or diluent may include time delay material well known to the art, such as glyceryl mono-stearate or glyceryl distearate alone or with a wax. The pharmaceutical composition according to the present invention can be administered in various manners to achieve the desired effect. Said pharmaceutical compositions can be administered either alone or in the formulated as pharmaceutical preparations to the subject being treated either orally, topically or parenterally. Moreover, the substance can be administered in combination with other substances either in a common pharmaceutical composition or as separated pharmaceutical compositions. The diluent is selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. A therapeutically effective dose refers to that amount of the substance according to the invention which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined, as set forth before, by the attending physician based on clinical factors. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. A typical doses were specified above. The pharmaceutical compositions and formulations referred to herein are administered at least once in accordance with the use of the present invention. However, the said pharmaceutical compositions and formulations may be administered more than one time, for example from one to four times daily up to a non-limited number of days. Specific formulations according to the invention are prepared in a manner well known in the pharmaceutical art and usually comprise at least one active substance referred to herein above in admixture or otherwise associated with a pharmaceutically acceptable carrier or diluent thereof. For making those formulations the active substance(s) will usually be mixed with a carrier or diluted by a diluent, or enclosed or encapsulated in a capsule, sachet, cachet, paper or other suitable containers or vehicles. A carrier may be solid, semisolid, gel-based or liquid material which serves as a vehicle, excipient or medium for the active ingredients. Said suitable carriers comprise those mentioned above and others well known in the art, see, e.g., Remington's Pharmaceutical Sciences, Mack Publishing Company, Easton, Pennsylvania. The formulations can be adopted to the mode of administration comprising the forms of tablets, capsules, suppositories, solutions, suspensions or the like. The dosing recommendations will be indicated in product labeling by allowing the prescriber to anticipate dose adjustments depending on the considered patient group, with information that avoids prescribing the wrong drug to the wrong patients at the wrong dose. Also preferred is the use of the invention, wherein said composition is a cosmetic composition. The term “cosmetic composition” relates to a composition which can be formulated as described for a pharmaceutical composition above. However, instead of pharmaceutical acceptable formulations, carriers diluents and the like the carries, diluents and excipients must be cosmetically acceptable. Moreover, cosmetic compositions are preferably to be applied topically. In a further preferred embodiment of the use of the invention said composition is formulated as a hair tonic, a hair restorer composition, a shampoo, a powder, a jelly, a hair rinse, an ointment, a hair lotion, a paste, a hair cream, a hair spray and/or a hair aerosol. Further preferably, said composition is to be administered topically to the skin or scalp of a subject. Topical administration is described in detail in US2003114526 the disclosure of which is hereby incorporated by reference. In a more preferred embodiment of the use of the invention said subject is a mammal. Most preferably, said mammal is a human, a dog, a cat, a horse, a rabbit, a sheep, a camel, a mouse, a rat, an alpaca, a vicuna, a guanaco or a lama. In a more preferred embodiment of the use of the invention said subject suffers from genetically determined and/or acquired forms of hair loss. Most preferably, said genetically determined or acquired form of hair loss is alopecia areata, alopecia subtotalis, alopecia totalis, trichotillomania or drug induced alopecia. The symptoms accompanied with these disease are well known to the physician and are described in detail in medical standard text books such as Stedman or Pschyrembel. The present invention also relates to a transgenic non-human animal comprising a nucleic acid as defined hereinabove encoding an IL-15 polypeptide, wherein said nucleic acid is specifically expressed in the keratinocytes of the hair bulb, in the Langerhans cells, the melanocytes, the dendritic epidermal T-cells, mast cells, cutaneous nerve fibres or fibroblasts. The term “transgenic non-human animal” relates to animals, preferably mammals, and cells of such animals which contain (preferably stably integrated into their genome) at least one of the aforementioned nucleic acids. Said nucleic acid is preferably linked to a suitable regulatory element which enables specific expression in the aforementioned tissues. This can be achieved either by introducing a transgene comprising the IL-15 nucleic acid and a nucleic acid encoding the regulatory sequence into the genome or by introducing the IL-15 nucleic acid specifically downstream of the endogenous regulatory element present in said animal (“knock in” animals). If a transgene comprising a regulatory sequence is inserted, said transgene is preferably integrated stably into the genome of the host animal. The trangene or the knock in constructs are designed as to enable sufficient expression of the IL-15 polypeptide encoded by the IL-15 nucleic acid. Suitable regulatory elements are preferably the langerin-promoter which is specific for Langerhans cells (Valladeau, Immunity 2000, 12 (1): 71-81), the tyrosinase-promoter which is specific for melanocytes (Kelsall, Cancer Res. 1998, 58: 4061-65), or the collagen-i alpha 2-promoter which directs expression to fibroblasts (Hibbard, Cancer Res. 2000, 60 (17): 4862-4872). A method for the production of a transgenic non-human animal, for example transgenic mouse, comprises introduction of a IL-15 nucleic acid or targeting vector into a germ cell, an embryonic cell, stem cell or an egg or a cell derived therefrom. Production of transgenic embryos and screening of those can be performed, e.g., as described by A. L. Joyner Ed., Gene Targeting, A Practical Approach (1993), Oxford University Press. The DNA of the embryonal membranes of embryos can be analyzed using, e.g., Southern blots with an appropriate probe; see supra. A general method for making transgenic non-human animals is described in the art, see for example WO 94/24274. For making transgenic non-human organisms including non-human animals obtained by homologous recombination, embryonal stem cells (ES cells) are preferred. Murine ES cells, such as AB-1 line, grown on mitotically inactive SNL76/7 cell feeder layers (McMahon and Bradley, Cell 62: 1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al., Nature 326: 292-295 (1987)), the D3 line (Doetschman et al., J. Embryol. Exp. Morph. 87: 27-45 (1985)), the CCE line (Robertson et al., Nature 323: 445-448 (1986)), the AK-7 line (Zhuang et al., Cell 77: 875-884 (1994) which is incorporated by reference herein). The success of generating a mouse line from ES cells bearing a specific targeted mutation depends on the pluripotency of the ES cells, i.e. their ability, once injected into a host developing embryo, such as a blastocyst or morula, to participate in embryogenesis and contribute to the germ cells of the resulting animal. The blastocysts containing the injected ES cells are allowed to develop in the uteri of pseudopregnant nonhuman females and are born as chimeric mice. The resultant transgenic mice are chimeric for cells having either the recombinase or reporter loci and are backcrossed and screened for the presence of the correctly targeted transgene by PCR or Southern blot analysis on tail biopsy DNA of offspring so as to identify transgenic mice heterozygous for either the recombinase or reporter locus/loci. A method for making an IL-15 transgenic non-human animal is described in detail in the accompanied Examples. The transgenic animals of the present invention can be advantageously used for the manufacture of hair to be used for industrial goods. Suitable candidates for transgenic animals are, therefore, a dog, a cat, a horse, a rabbit, a sheep, a camel, a mouse, a rat, an alpaca, a vicuna, a guanaco or a lama.

The present invention, accordingly, also relates to a method for stimulating hair growth in a non-human animal comprising the steps of: (a) Transforming said animal with a nucleic acid as defined in claim 1; and (b) Expressing the IL-1 5 polypeptide encoded by said nucleic acid. The term “Transforming” as used herein relates to all techniques for introducing a transgene into an animal as disclosed before. In addition said term encompasses methods for introducing a nucleic acid to be expressed topically, e.g., on the skin or scalp. This may be achieved by using gene therapeutic approaches disclosed above. The term “Expressing the IL-15 polypeptide” encompasses all means such as stimulation, induction etc. necessary for allowing production of IL-15 polypeptides in said animal. Depending on the regulatory elements used to control the expression of the nucleic acid, expression will occur permanently or might require induction or stimulation. Such a stimulus could be UV radiation. Moreover, several inducible regulators elements for governing gene expression are well known in the art. These elements can be induced by, e.g., heat shock, dexamethasone, or metal ions. Preferred regulatory elements will be disclosed in detail below.

In addition, the present invention encompasses a method for manufacturing animal hair comprising the steps of:
(a) Transforming said animal with a nucleic acid as defined in claim 1; and
(b) Expressing the IL-15 polypeptide encoded by said nucleic acid.

It should be understood that the term “manufacturing” as used herein comprises in addition to the method having the steps as set forth above methods which may comprise further steps known to be required for the manufacture of hair.

In a preferred embodiment of the methods of the invention said IL-15 polypeptide is expressed under the control of a regulatory element.

The term “regulatory element” as used herein refers to nucleic acid sequences which either control expression of a DNA molecule or the translation of an RNA molecule. Such regulatory elements are usually derived from naturally occurring regulatory elements of genes. It is well known in the art that genes comprise structural elements which encode an amino acid sequence as well as regulatory elements which are involved in the regulation of the expression of said genes. Structural elements are represented by exons which may either encode an amino acid sequence or which may encode for RNA which is not encoding an amino acid sequence but is nevertheless involved in RNA function, e.g. by regulating the stability of the RNA or the nuclear export of the RNA. Regulatory elements of a gene may comprise promoter elements or enhancer elements both of which could be involved in transcriptional control of gene expression. It is very well known in the art that a promoter is to be found upstream of the structural elements of a gene. Regulatory elements such as enhancer elements, however, can be found distributed over the entire locus of a gene. Said elements could be reside, e.g., in introns, regions of genomic DNA which separate the exons of a gene. Promoter or enhancer elements correspond to polynucleotide fragments which are capable of attracting or binding polypeptides involved in the regulation of the gene comprising said promoter or enhancer elements. For example, polypeptides involved in regulation of said gene comprise the so called transcription factors. Said introns may comprise further regulatory elements, which are required for proper gene expression. Introns are usually transcribed together with the exons of a gene resulting in a nascent RNA transcript which contains both, exon and intron sequences. The intron encoded RNA sequences are usually removed by a process known as RNA splicing. However, said process also requires regulatory sequences present on a RNA transcript said regulatory sequences may be encoded by the introns. In addition, besides their function in transcriptional control and control of proper RNA processing and/or stability, regulatory elements of a gene could be also involved in the control of genetic stability of a gene locus. Said elements control, e.g., recombination events or serve to maintain a certain structure of the DNA or the arrangement of DNA in a chromosome.

More preferably, said regulatory element enables specific expression in the keratinocytes of the hair bulb, in the Langerhans cells, the melanocytes, the dendritic epidermal T-cells, mast cells, cutaneous nerve fibres or fibroblasts. Such a specific expression can be achieved by expressing the IL-15 polypeptide of the invention under the control of a suitable regulatory element. Such elements are preferably the langerin-promoter which is specific for Langerhans cells, the tyrosinase-promoter which is specific for melanocytes, or the collagen-i alpha 2-promoter which directs expression to fibroblasts.

In another more preferred embodiment of the methods of the invention said methods further comprise the step of administering to the skin and/or scalp of a non-human animal the composition of the invention as defined hereinabove.

The present invention also encompasses a method for manufacturing animal hair comprising the step of administering to the skin and/or scalp of a non-human animal the composition of the invention as defined hereinabove.

In furthermore preferred embodiment of the methods of the invention said methods further comprising the step of obtaining the hair of said animal. The term “obtaining” as used herein refers to methods for separating the hair from said animal. This could be achieved by either separating the hair from the skin or scalp of the animal, e.g. by shaving, or by separating the skin or scalp including the hair from said animal. Depending on the purpose for which the hair is to be used and the animal the person skilled in the art will select a suitable method without further ado. Most preferably, the animal referred to hereinabove is a dog, a cat, a horse, a rabbit, a sheep, a camel, a mouse, a rat, an alpaca, a vicuna, a guanaco or a lama.

The present invention, moreover, encompasses a method for manufacturing an inhibitor of IL- 15 as specified hereinabove comprising the step of determining an inhibitor of at least one or preferably all biological activities of IL-15 polypeptides. Such determination of an inhibitor can be carried out using an assay for determination of IL-15 inhibitor comprising the following steps: (a) contacting cells known to be responsive to IL-15, preferably keratinocytes, NK or NKT cells, T cells, antigen-presenting cells or lymphoid or mesenchymal cells with IL-15 polypeptide and a potential inhibitor, and (b) determining the response of said cells upon administration of the potential inhibitor whereby an inhibitor prevents the biological response induced by IL-15. Preferred responses to be determined are stimulation of growth in the case of keratinocytes, T cells, NK cells or NKT cells or prevention of programmed cell death upon induced by an apoptotic stimulus in the case of lymphoid or mesenchymal cells or activation of antigen-presenting function. Said responses can be determined by administering to said cells IL-15 polypeptide only. The details of such an assay have been disclosed elsewhere in this specification. Alternatively, an inhibitor can be determined by an assay comprising the steps of (a) contacting a specific IL-15 antibody or the IL-15 receptor alpha chain with IL-15 and a potential and (b) determining competition between IL-15 and said inhibitor and for antibody or receptor binding. The inhibitor identified by said assay must not have IL-1 5 activity. Preferably, the inhibitor has no biological activity at all besides inhibiting/competing with IL-15 polypeptides. For determination of the competition it is preferred that either the potential inhibitor or the IL-15 will be linked to a detectable label, such as a radioisotope or a chromogenic chemical entity. Suitable molecules which could be used in the screening assays referred to hereinabove are preferably those which are chemical entities to be screened for candidate compounds according to the invention. Moreover, the method of manufacturing an inhibitor comprises preferably the further step of chemically or biologically formulating the inhibitor determined as specified hereinabove. The means for formulation depend on the chemical nature of the identified inhibitor. For instance, chemical entities such as small organic molecules or short peptides can be synthesized by well known techniques described in standard text books of biochemistry or chemistry. Biological molecules such as polypeptides, antibodies etc. can be manufactured by standard molecular biology techniques. The formulation may also encompass further steps of pharmaceutical or cosmetic formulation in accordance with the required standards such as good manufacturing practice (GMP) or comparable standards.

Accordingly, the invention also relates to a method for the manufacture of a pharmaceutical or cosmetic composition comprising the steps of determining and manufacturing the IL-15 polypeptide inhibitor as specified above and formulating said inhibitor in a pharmaceutically or cosmetically acceptable form optionally with a pharmaceutical or cosmetic carrier as specified elsewhere in this specification.

Encompassed by the present invention is the use of such IL-15 inhibitors for the preparation of a pharmaceutical composition for inhibiting hair growth. Such hair growth may be caused by a disease condition resulting in an IL-15 overexpression in the skin of subjects suffering from said disease condition. Inhibitors of IL-15 to be used for inhibiting hair growth fuirther comprise antibodies, including those types defined elsewhere in this specification, which are capable of specifically recognizing IL-15 polypeptiles, antisense RNA molecules or small interfering RNA molecules (RNAi).

Finally, the present invention relates to a method of treating, preventing and/or ameliorating a subject which suffers from hair loss comprising the step of administering a composition of the invention as defined hereinabove in an effective dosage to said subject. All embodiments specified in connection with the uses of the present invention apply for said method mutatis mutandis.

Several documents are cited throughout this specification either by name or are referred to by numerals in parenthesis. Full bibliographic citations are found below. The relevant disclosure of each of the documents cited herein including any manufacturer's specifications, instructions, etc. is hereby incorporated by reference.

### THE FIGURES SHOW

FIG. 1: Generation of IL-15 tg mice. The K14-IL-15 expression cassette bearing the human K14 promoter (white box), a rabbit 13-globin intron (grey box), the mIL-15 cDNA fused to the CD33 signal peptide and the FLAG epitope tag (black box) as well as the K14 polyadenylation site (polyA, hatched box) was used for microinjection.

FIG. 2: (A) Immunohistochemical staining of ear skin using an anti-FLAG Ab showed transgene expression in basal keratinocytes (indicated by black arrows). (B) Immunoblot of skin, serum and thymus for IL-15. Equal amounts of total protein were loaded and analyzed using an anti-murine IL- 15 Ab.

FIG. 3: Increased hair growth in tg mice. Littennate controls (left) and IL- 15 tg mice (right) were shaved on the back, and 6 days later hair growth was documented by digital photography.

FIG. 4: Inproved de novo development of hair follicles in IL-15-tg mice. Littermate controls (left) and IL-15 tg mice (right) were depilated on the back, and 14 days later hair growth was documented by digital photography (A). Hair follicle differentiation (indicated by black arrows) in tg mice demonstrated by hematoxylin-eosin stained histology sections of the skin from wildtype and IL-15 tg mice 14 days after depilation (B).

FIG. 5: Nucleic acid sequence of modified mIL-15 (SEQ ID NO:7).

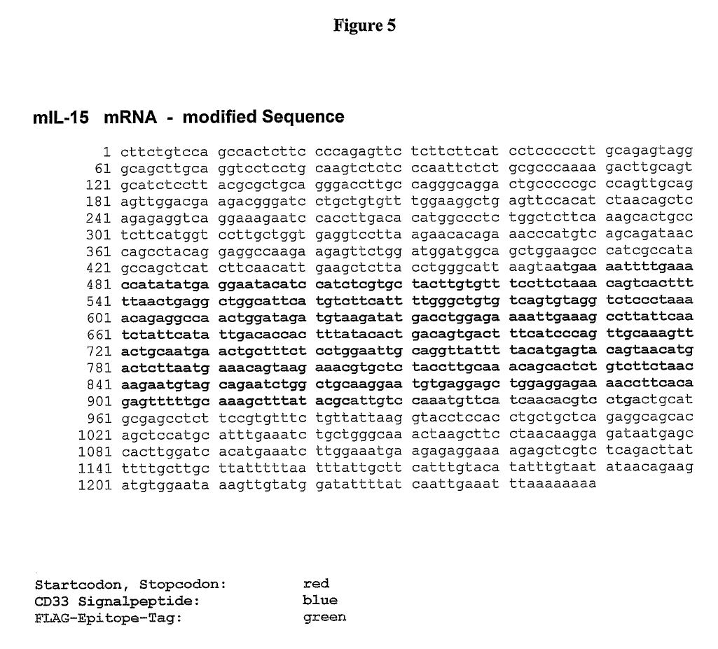

### The invention will now be described by reference to the following biological Examples which are merely illustrative and are not constructed as a limitation of the scope of the present invention.

### Example 1

### Basic methods

Generation ofIL-15 transgenic mice

The gene for murine IL-15 was placed under the control of the human K14 promoter using standard methods as follows: The K14 expression cassette used included the K14 promoter, a rabbit B-globin intron, the BamHI cloning site and the K14 polyadenylation site (22,23). The BamHI cloning site was modified by ligating a polylinker into this site resulting in a multiple cloning site containing the restriction enzyme sites SalI, BglIl, BamHI and Xbal resulting in the plasmid pAMM1 1. The approximately 500 bp BglII/XbaI fragment of CD33 IL-15 FLAG was cloned into the BgIII/XbaI site of pAMM1 1 to create pAMM77.

Plasmid DNA to be used for microinjections was first purified through CsCl gradient centrifugation. The expression cassette containing the IL-15 gene was released from the plasmid and purified through 0.7% agarose gel electrophoresis following digestion with SphI and Smal, extracted from the gel (PCR purification kit; Roche, Mannheim, Germany), resuspended in TE* buffer (10 mM Tris pH 7.4/0.1 mM EDTA) and used for microinjection at a concentration of 2 ng/jl into mouse C57BL/6/C3H/HeN F, x C57BL/6 and FVB/N oocytes. Two founder lines with similar transgene expression were identified by either PCR (AM28: CAATGATATACACTGTTTGAGATGA (SEQ ID NO: 5); AM65: CGTGTTGATGAACATTTGGACAA (SEQ ID NO: 6); Cycling profile: 95 ° C for 3 min; [95 ° C for 1 min; 54 ° C for 1 min; 72 ° C for 1 min x 35; 72 ° C for 5 min] and Southern blotting. Experiments were performed with tg mice on a C57BL/6/C3H/HeN background. Mice were housed under specific pathogen-free (spf) conditions and experiments were performed according to institutional regulations.

Depilation and Shaving

IL-15 transgenic (IL-15 tg) mice and wildtype controls were anesthetized with 1 % Ketamin (1.5 ,ul per g body weight; Merck, Darmstadt, Germany), and then the back was shaved using an electric animal shaver. For depilation hairs were plucked out with forceps. Mice were observed for hair growth daily, and results were documented by digital photography. All mice were used at 8-12 weeks of age.

Histopathological Analysis

For light microscopy, punch biopsies (8 mm in diameter) of murine skin were fixed by immersion in 10 % neutral buffered formaline. Subsequently tissues were dehydrated with ethanol, transferred to xylene, and embedded in paraffin using standard histology techniques, and 5-tim sections were cut and stained with hematoxylin and eosin. Tissue sections were mounted onto slides and analyzed using an Olympus BX61 microscope and the MetaMorph software (Visitron Systems, Puchheim, Germany).

Immnunohistology

Inmunuhistochemistry was performed on cryostat sections of ears (6 - 8 tm) fixed in acetone according to standard methods (24,25). Sections were blocked with 0.5 % bovine serum albumin (Sigma, Tauflkirchen, Germany) in PBS, then incubated in the appropriate dilutions of monoclonal antibodies or isotype control and subsequently incubated with a horseradish peroxidase (HRP)-coupled secondary antibody. Peroxidase activity was visualized using 3- amino-9-ethyl-carbazol as a chromogen. Tissues were counterstained with MAYER'S hemalaun solution (Merck, Darmstadt, Germany). Biotinylated mouse anti-FLAG was obtained from Sigma. Sections were examined using an Olympus BX61 microscope and the MetaMorph software.

Western Blot Analysis

Serum of IL-15 tg mice and wildtype controls as well as skin and thymus lysates were loaded directly onto 15 % polyacrylamid gels. Recombinant mIL- 15 protein was used as a positive control. Proteins were electrophoresed under denaturing conditions and electroblofted onto nitrocellulose membranes (Amersham Biosciences Europe, Freiburg, Germany) at 150 mA for 1 h. Membranes were blocked for 2 h with 5 % non-fat dry milk in TBS plus 0.5 % Tween 20 (TBST) and then incubated overnight with the appropriate antibody (anti-mouse IL-15, clone M49, a kind gift from Genmab, Utrecht, The Netherlands) diluted 1:500 in TBST plus 1% non-fat dry milk. Membranes were washed with TBST and incubated for 2 h with horseradish peroxidase-conjugated secondary antibody diluted 1:1000 in TBST. Proteins were detected using enhanced chemoluminescence reagents (Amersham Pharmacia Biosciences, Europe).

### Example 2

### Phenotype of IL-15 tg mice

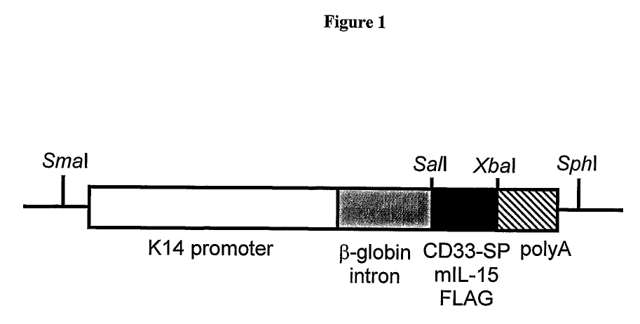

To investigate the role of keratinocyte-derived IL-1 5 in vivo, IL-1 5 expression was targeted to the epidermis using the K14 expression cassette shown in FIG. 1. Several regulatory posttranscriptional mechanisms have been identified, which control IL-15 translation and secretion (17, 19). Therefore, a modified mouse IL-15 cDNA was cloned that lacks the posttranscriptional checkpoints controlling endogenous IL-15 production, thus yielding optimal IL-15 overexpression and secretion. Modifications of the transgenic construct are depicted in FIG. 1 and included removing upstream AUGs that impair translation, replacing the insufficiently translated and secreted endogenous IL-15 signal peptide with the CD33 signal peptide, and stabilizing the COOH terminus of the IL-1 5 protein with a FLAG epitope tag.

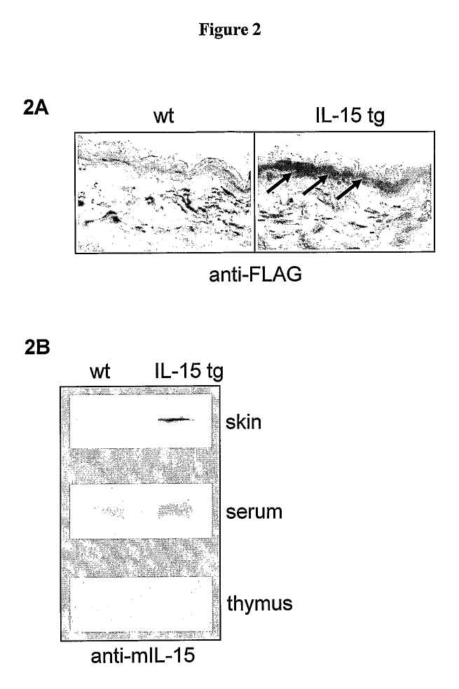

This FLAG epitope tag also enabled the differentiation between endogenous and tg IL-15 expression in the skin. The K14 promoter drives gene expression of most basal cells of stratified squamous epithelium including the epidermis. To show correct expression of the transgene, immunohistochemistry and Western blot analysis was performed on tg skin tissue using an anti-FLAG and anti-IL-15 Ab. As demonstrated in FIG. 2A a strong and uniform expression of IL-1 5/FLAG was detected in the epidermis of tg animals. IL-15 protein can be detected in the serum of transgenic mice using an anti-IL-15 Ab (FIG. 2B). Transgene expression followed the expected pattern in other tissues, with the exception that IL-15 expression was not detectable in the thymus, where K14 expression has been documented in some transgenic strains before (22). The transgene insertion locus or possibly the transgene copy number may account for the lack of IL-15 expression in the thymus. Homozygous tg mice breed well and are healthy even over an observation period of 15 months.

### Example 3

### Enhanced Hair Growth in tg Mice After Shaving and Depilation

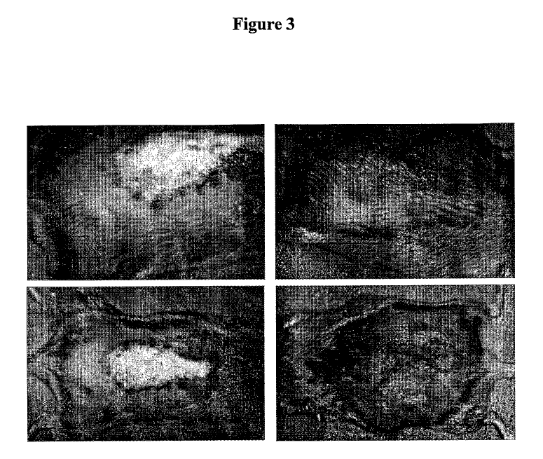

Since IL-1 5 has been reported to mediate cell survival and to act as a growth factor for mast cells (26,27) we hypothesized that keratinocyte derived IL-15 in tg mice might also have stimulatory effects on hair follicles. To evaluate if cutaneous IL-15 overexpression induced hair growth in vivo groups of littermate and tg animals were shaved on their back and hair growth was observed daily for two weeks. In contrast to litterinate controls, IL-15 tg mice showed a significantly enhanced hair growth within 6 days (FIG. 3) indicating that efficiently secreted IL-15 in tg mice leads to activation of hair follicle cells and as a consequence to improved hair growth.

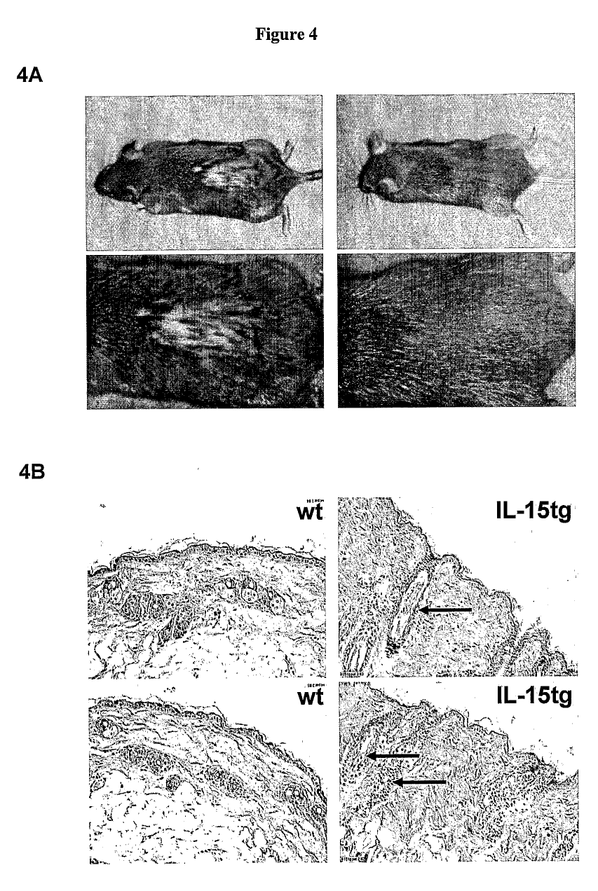

We next addressed whether plucking out tufts of hair and thereby removing the hair shafts completely also leads to increased hair growth in IL-15 tg mice. To this end, wildtype and IL- 15 tg mice were anesthetized and depilated using forceps. Both groups of animals were observed daily for hair growth. As demonstrated in FIG. 4A IL-15 tg mice showed a completely reconstituted hair covering already 14 days after depilation in contrast to controls. In addition to the results shown previously (FIG. 3) this demonstrates that secreted IL-15 in tg mice may not only be responsible for the activation of undisturbed hair follicles but perhaps is also needed for the in vivo de novo development. To prove this assumption we prepared 8 mm punch biopsies from depilated skin areas of IL-15 tg and wildtype mice 14 days after depilation. The histopathological analysis of this paraffin embedded hematoxylin-eosin stained skin sections revealed multiple morphologically intact hair follicles in IL-15 tg epidermis whereas in wildtype skin the hair follicles are not completely restored (FIG. 4B). These findings indicate that IL-15 has tremendous effects on the activation of hair follicle cells leading to enhanced hair growth and is also absolutely necessary for the de novo synthesis of hair follicles.

### REFERENCES

1. Grabstein, K.H., J. Eisenman, K. Shanebeck, C. Rauch, S. Srinivasan, V. Fung, C. Beers, J. Richardson, M.A. Schoenborn, M. Ahdieh, L. Johnson, M.R. Alderson, J.D. Watson, D.M. Anderson, and J.G. Giri. 1994. Cloning of a T cell growth factor that interacts with the B chain of the interleukin-2 receptor. Science 264: 965.
2. Zhang, X., S. Sun, I. Hwang, D.F. Tough, and J. Sprent. 1998. Potent and selective stimulation of memory-phenotype CD8+T cells in vivo by IL- 15. Immunity 8: 591.
3. Waldmann, T.A., Y. Tagaya. 1999. The multifaceted regulation of interleukin-15 expression and the role of this cytokine in NK cell differentiation and host response to intracellular pathogens. Ann. Rev. Immunol. 17:19.
4. Waldmann T.A., S. Dubois, and Y. Tagaya. 2001. Contrasting roles of IL-2 and IL-15 in the life and death of lymphocytes: implications for immunotherapy. Immunity 14: 105.
5. Kennedy, M.K., M. Glaccum, S.N. Brown, E.A. Butz, J.L. Viney, M. Embers, N. Matsuki, K. Charrier, L. Sedger, C.R. Willis, K. Brasel, P.J. Morrissey, K. Stocking, J.C.L. Schuh, S. Joyce, and J.J. Peschon. 2000. Reversible defects in natural killer and memory CD8 T cell lineages in interleukin-1 5-deficient mice. J Exp. Med. 191: 771.
6. Fehniger, T.A., K. Suzuki, A. Ponnappan, J.B. VanDeusen, M.A. Cooper, S.M. Florea, A.G. Freud, M.L. Robinson, J. Durbin, and M.A. Caligiuri. 2001. Fatal leukemia in interleukin-15 transgenic mice follows early expansion in natural killer and memory phenotype CD8+T cells. J Exp. Med. 193: 219.
7. Hayday, A., E. Theodoridis, E. Ramsburg, and J. Shires. 2001. Intraepithelial lymphocytes: exploring the third way in immunology. Nat. Immunol. 2: 997.
8. Dooms, H., M. Desmedt, S. Vancaeneghem, P. Rottiers, V. Goossens, W. Fiers, and J. Grooten. 1998. Quiescence-inducing and anti-apoptotic activities of IL-15 enhance secondary CD4+T cell responsiveness to antigen. J Immunol. 161: 2141.
9. Ye, S.-K., K. Maki, H.-C. Lee, A. Ito, K. Kawai, H. Suzuki, T.W. Mak, Y.-H. Chien, T. Honjo, and K. Ikuta. 2001. Differential role of cytokine receptors in the development epidermal y6 T cells. J: Immunol. 167: 1929.
10. De Creus, A., K. Van Beneden, F. Stevenaert, V. Debacker, J. Plum, and G. Leclercq. 2002. Developmental and functional defects of thymic and epidermal V gamma 3 cells in IL- 15-deficient and IFN regulatory factor-l-deficient mice. J Immunol. 168: 6486.
11. Lodolce, J. P., D.L. Boone, S. Chai, R.E. Swain, T. Dassopoulos, S. Trettin, and A. Ma. 1998. IL-15 receptor maintains lymphoid homeostasis by supporting lymphocyte homing and proliferation. Immunity 9:669.
12. Quinn, L.S., K.L. Haugk, and K.H. Grabstein. 1995. Interleukin-15: a novel anabolic cytokine for skeletal muscle. Endocrinology 136: 3669.
13. Tagaya, Y., J.D. Burton, Y. Miyamoto, and T.A. Waldmann. 1996. Identification of a noval receptor/signal transduction pathway for IL-1 5/T in mast cells. EMBO J 15: 4928.
14. Bulfone-Paus, S., D. Ungureanu, T. Pohl, G. Lindner, R. Paus, R. Rutckert, H. Krause, and U. Kunzendorf. 1997. Interleukin-15 protects from lethal apoptosis in vivo. Nat. Med. 3: 1124.
15. Rutckert, R., K. Asadullah, M. Seifert, V.M. Budagian, R. Arnold, C. Trombotto, R. Paus, and S. Bulfone-Paus. 2000. Inhibition of keratinocyte apoptosis by IL-1 5: A new parameter in the pathogenesis of psoriasis? J Immunol. 165: 2240.
16. Reinecker, H.-C., R.P. MacDermott, S. Mirau, A. Dignass, and D. Podolsky. 1996. Intestinal epithelial cells both express and respond to interleukin 15. Gastroenterology 111: 1706.
17. Bamford, R.N., J.D. Battiata, J.D. Burton, H. Sharma, and T.A. Waldmann. 1996. Interleukin(IL)-15/IL-T production by the adult T-cell leukemia cell line HuT-102 is associated with a human T-cell lymphotrophic virus type I region/IL-15 fusion mesage that lacks many upstream AUGs that normally attenuates IL-15 mRNA translation. Proc. Natl. Acad. Sci. USA 93: 2897.
18. Onu, A., T. Pohl, H. Krause, and S. Bulfone-Paus. 1997. Regulation of IL-15 secretion via the leader peptide of two IL-15 isoforms. J ImmunoL. 158: 255.
19. Tagaya, Y., G. Kurys, T.A. Thies, J.M. Losi, N. Azimi, J.A. Hanover, R.N. Bamford, and T.A. Waldmann. 1997. Generation of secretable and nonsecretable interleukin 15 isoforms through altemat usage of signal peptides. Proc. Natl. Acad. Sci. USA 94: 14444.
20. Mohamadzadeh, M., A. Takashima, I. Dougherty, J. Knop, P.R. Bergstresser, and P.D. Cruz, Jr. 1995. Ultraviolet B irradiation up-regulates the expression of IL-15 in human skin. J Immunol. 155: 4492-4496.
21. Blauvelt, A., H. Asada, V. Klaus-Kovtun, D.J. Altman, D.R. Lucey, and S.I. Katz. 1996. Interleukin-15 MRNA is expressed by keratinocytes, Langerhans cells, and blood-derived dendritic cells and is down-regulated by ultraviolet B irradiation. J Invest. Dermatol. 106: 1047.
22. Vassar, R., M. Rosenberg, E. Ross, A. Tyner, and E. Fuchs. 1989. Tissue-specific and differentiation-specific expression of a human K14 keratin gene in transgenic mice. Proc. Natl. Acad. Sci. USA 86: 1563.
23. Saitou, M., S. Sugai, T. Tanaka, K. Shimouchi, E. Fuchs, S. Narumiya, and A. Kakizuka. 1995. Ihibition of skin development by targeted expression of a dominant-negative retinoic acid receptor. Nature 374: 159.
24. Grabbe, S., K. Steinbrink, M. Steinert, T.A. Luger, and T. Schwarz. 1995. Removal of the majority of epidermal Langerhans cells by topical or systemic steroid application enhances the effector phase of murine contact hypersensitivity. J Immunol 155:4207.
25. Mehling, A., K. Loser, G. Varga, D. Metze, T.A. Luger, T. Schwarz, S. Grabbe, and S. Beissert. 2001. Overexpression of CD40 ligand in murine epidermis results in chronic skin inflammation and systemic autoimmunity. J Exp. Med. 194: 615.
26. Ku, C.C., M. Murakami, A. Sakamoto, J. Kappler, and P. Marrack. 2000. Control of homeostasis of CD8+T cells by opposing cytokines. Science 288: 675.
27. Judge, A.D., X. Zhang, H. Fuji, C.D. Surh, and J. Sprent. 2002. Interleukin-15 controls both proliferation and survival of a subset of memory-phenotype CD8+T cells. J Exp. Med. 196: 935-946.
28. Liew, F.Y. 2003. The role of innate cytokines in inflammatory response. Immunol. Lett. 85: 131-134
29. Porter, R.M. 2003. Mouse models for human hair loss disorders. J Anat. 202: 125-131
30. Mutller-Rover, S., H. Rossiter, G. Lindner, E.M.J. Peters, T.S. Kupper and R. Paus. 1999. Hair follicle apoptosis and Bcl-2. J Invest. Dermatol. Sympos. Proc. 4: 272-277
31. Reddy S., T. Andl, A. Bagasra, M.M. Lu, D.J. Eppstein, E.E. Morrisey. 2001. Characterisation of wnt gene expression in developing and postnatal hair follicles and identification of wnt5a as a target of sonic hedgehog in hair follicle morphogenesis. Mech. Dev. 107: 69-82
32. Stenn K.S., R. Paus. 2001. Controls of hair follicle cycling. Physiol. Rev. 81: 449-494
33. Ruickert R., G. Lindner, S. Bulfone-Paus, R. Paus. 2000. High-dose proinflammatory cytokines induce apoptosis of hair bulb keratinocytes in vivo. British J Dermatology 143: 1036-1039
34. Lindner G., VA Botchkarev, NV Botchkarev et al. 1997. Analysis of apoptosis during hair follicle regression. Am. J Pathol. 151: 1601-17

1-23. (canceled)

24. A method for stimulating hair growth comprising administering to a subject in a therapeutically effective amount of a composition comprising:
(i) a polynucleotide comprising
(a) a nucleic acid sequence as shown in SEQ ID NO: I or 3,
(b) a nucleic acid sequence encoding an amino acid sequence as shown in SEQ ID NO: 2 or 4,
(c) a nucleic acid sequence encoding an amino acid sequence as shown in SEQ ID NO: 2 or 4 having a modified signal peptide, a modified N- terminus and/or a modified C-terminus, or
(d) a nucleic acid sequence which hybridises under stringent conditions to any one of (a) to (c);
(ii) a polypeptide encoded by the nucleic acid as defined in any one of (a) to (c); or
(iii) a compound which binds to an antibody which specifically recognizes the polypeptide defined in (ii) or which specifically binds to an IL- 15 receptor alpha chain.

25. The method of claim 24, wherein said composition further comprises a second hair growth stimulating agent.

26. The method of claim 25, wherein said second hair growth stimulating agent is selected from the group consisting of a zinc salt of a carboxylic acid, a saponin, a triterpene, crataegolic acid, celastrol, Asiatic acid, an inhibitor of 5-[alpha]- reductase, a 1,4-methyl-4-azasteroid, an androgen receptor antagonist, cyclosporin, triiodothyronine, diazoxide, a potassium channel opener, a derivative of oestrogen; and mixtures thereof.

27. The method of claim 26, wherein the second hair growth stimulating agent is a triterpene and is at least one of oleanolic acid and ursolic acid.

28. The method of claim 26, wherein the second hair growth stimulating agent is an inhibitor of 5-[alpha]-reductase which is progesterone.

29. The method of claim 26, wherein the second hair growth stimulating agent is a 1,4-methyl-4-azasteroid which is 17-[beta]-N,N-diethylcarbamoyl-4-methyl-4- aza-5-[alpha]-androstan-3-one.

30. The method of claim 26, wherein the second hair growth stimulating agent is an androgen receptor antagonists which is one or more of cyproterone acetate, MINOXIDIL®, azaelaic acid and derivatives thereof.

31. The method of claim 26, wherein the second hair growth stimulating agent is a potassium channel opener which is one or more of cromakalin, phenytoin, and mixtures thereof.

32. The method of claim 26, wherein the second hair growth stimulating agent is a derivative of oestrogen which is oestradiolvalerate.

33. The method of claim 24, wherein said composition further comprises a pharmaceutically or cosmetically acceptable carrier.

34. The method of claim 24, wherein said composition is a pharmaceutical composition.

35. The method of claim 24, wherein said composition is a cosmetic composition.

36. The method of claim 24, wherein said composition is formulated as a hair tonic, a hair restorer composition, a shampoo, a powder, a jelly, a hair rinse, an ointment, a hair lotion, a paste, a hair cream, a hair spray and/or a hair aerosol.

37. The method of claim 24, wherein said composition is administered topically to the skin or scalp of a subject.

38. The method of claim 37, wherein said subject is a mammal.

39. The method of claim 38, wherein said mammal is a human, a dog, a cat, a horse, a rabbit, a sheep, a camel, a mouse, a rat, an alpaca, a vicuna, a guanaco or a lama.

40. The method of claim 37, wherein said subject suffers from genetically determined and/or acquired form of hair loss.

41. The method of claim 40, wherein said genetically determined or acquired form of hair loss is alopecia areata, alopecia subtotalis, alopecia totalis, trichotillomania or drug induced alopecia.

42. A method for treating, preventing and/or ameliorating hair loss comprising administering to a subject in a therapeutically effective amount of a composition comprising:
(i) a polynucleotide comprising
(a) a nucleic acid sequence as shown in SEQ ID NO: I or 3,
(b) a nucleic acid sequence encoding an amino acid sequence as shown in SEQ ID NO: 2 or 4,
(c) a nucleic acid sequence encoding an amino acid sequence as shown in SEQ ID NO: 2 or 4 having a modified signal peptide, a modified N- terminus and/or a modified C-terminus, or
(d) a nucleic acid sequence which hybridises under stringent conditions to any one of (a) to (c);
(ii) a polypeptide encoded by the nucleic acid as defined in any one of (a) to (c); or
(iii) a compound which binds to an antibody which specifically recognizes the polypeptide defined in (ii) or which specifically binds to an IL-15 receptor alpha chain.

43. The method of claim 42, wherein said composition further comprises a second hair growth stimulating agent.

44. The method of claim 43, wherein said second hair growth stimulating agent is selected from the group consisting of a zinc salt of a carboxylic acid, a saponin, a triterpene, crataegolic acid, celastrol, Asiatic acid, an inhibitor of 5-[alpha]- reductase, a 1,4-methyl-4-azasteroid, an androgen receptor antagonist, cyclosporin, triiodothyronine, diazoxide, a potassium channel opener, a derivative of oestrogen; and mixtures thereof.

45. The method of claim 44, wherein the second hair growth stimulating agent is a triterpene and is at least one of oleanolic acid and ursolic acid.

46. The method of claim 44, wherein the second hair growth stimulating agent is an inhibitor of 5-[alpha]-reductase which is progesterone.

47. The method of claim 44, wherein the second hair growth stimulating agent is a 1,4-methyl-4-azasteroid which is 17-[beta]-N,N-diethylcarbamoyl-4-methyl-4- aza-5-[alpha]-androstan-3-one.

48. The method of claim 44, wherein the second hair growth stimulating agent is an androgen receptor antagonists which is one or more of cyproterone acetate, MINOXIDIL®, azaelaic acid and derivatives thereof.

49. The method of claim 44, wherein the second hair growth stimulating agent is a potassium channel opener which is one or more of cromakalin, phenytoin, and mixtures thereof.

50. The method of claim 44, wherein the second hair growth stimulating agent is a derivative of oestrogen which is oestradiolvalerate.

51. The method of claim 44, wherein said composition further comprises a pharmaceutically or cosmetically acceptable carrier.

52. The method of claim 44, wherein said composition is a pharmaceutical composition.

53. The method of claim 44, wherein said composition is a cosmetic composition.

54. The method of claim 44, wherein said composition is formulated as a hair tonic, a hair restorer composition, a shampoo, a powder, a jelly, a hair rinse, an ointment, a hair lotion, a paste, a hair cream, a hair spray and/or a hair aerosol.

55. The method of claim 44, wherein said composition is administered topically to the skin or scalp of a subject.

56. The method of claim 55, wherein said subject is a mammal.

57. The method of claim 56 wherein said mammal is a human, a dog, a cat, a horse, a rabbit, a sheep, a camel, a mouse, a rat, an alpaca, a vicuna, a guanaco or a lama.

58. The method of claim 55, wherein said subject suffers from genetically determined and/or acquired form of hair loss.

59. The method of claim 58, wherein said genetically determined or acquired form of hair loss is alopecia areata, alopecia subtotalis, alopecia totalis, trichotillomania or drug induced alopecia.

60. A transgenic non-human animal comprising a nucleic acid comprising
(a) a nucleic acid sequence as shown in SEQ ID NO: I or 3,
(b) a nucleic acid sequence encoding an amino acid sequence as shown in SEQ ID NO: 2 or 4,
(c) a nucleic acid sequence encoding an amino acid sequence as shown in SEQ ID NO: 2 or 4 having a modified signal peptide, a modified N- terminus and/or a modified C-terminus, or
(d) a nucleic acid sequence which hybridises under stringent conditions to any one of (a) to (c); wherein said nucleic acid is specifically expressed in the keratinocytes of the hair bulb, in the Langerhans cells, in the melanocytes, in the dendritic epidermal T-cells, in the mast cells, in cutaneous nerve fibres or in fibroblasts.

61. The transgenic non-human animal of claim 60, wherein said animal is a dog, a cat, a horse, a rabbit, a sheep, a camel, a mouse, a rat, an alpaca, a vicuna, a guanaco or a lama.

62. A method for stimulating hair growth in a non-human animal comprising:
(i) transforming said animal with a nucleic acid comprising
(a) a nucleic acid sequence as shown in SEQ ID NO: 1 or 3,
(b) a nucleic acid sequence encoding an amino acid sequence as shown in SEQIDNO: 2or4,
(c) a nucleic acid sequence encoding an amino acid sequence as shown in SEQ ID NO: 2 or 4 having a modified signal peptide, a modified N- terminus and/or a modified C-terminus, or
(d) a nucleic acid sequence which hybridises under stringent conditions to any one of (a) to (c);
(ii) expressing the polypeptide encoded by said nucleic acid.

63. The method of claim 62, wherein said IL-15 polypeptide is expressed under the control of a regulatory element.

64. The method of claim 63, wherein said regulatory element enables specific expression in the keratinocytes of the hair bulb, in the Langerhans cells, in the melanocytes, in the dendritic epidermal T-cells, in the mast cells, in cutaneous nerve fibres or in fibroblasts.

65. The method of claim 62, wherein said animal is a dog, a cat, a horse, a rabbit, a sheep, a camel, a mouse, a rat, an alpaca, a vicuna, a guanaco or a lama.

66. A method for manufacturing non-human animal hair comprising:
(i) transforming said non-human animal with a nucleic acid comprising
(a) a nucleic acid sequence as shown in SEQ ID NO: I or 3,
(b) a nucleic acid sequence encoding an amino acid sequence as shown in SEQ ID NO: 2 or 4,
(c) a nucleic acid sequence encoding an amino acid sequence as shown in SEQ ID NO: 2 or 4 having a modified signal peptide, a modified N- terminus and/or a modified C-terminus, or
(d) a nucleic acid sequence which hybridises under stringent conditions to any one of (a) to (c); and
(ii) expressing the polypeptide encoded by said nucleic acid.

67. The method of claim 66, wherein said IL-15 polypeptide is expressed under the control of a regulatory element.

68. The method of claim 67, wherein said regulatory element enables specific expression in the keratinocytes of the hair bulb, in the Langerhans cells, in the melanocytes, in the dendritic epidermal T-cells, in the mast cells, in cutaneous nerve fibres or in fibroblasts.

69. The method of claim 67, wherein said animal is a dog, a cat, a horse, a rabbit, a sheep, a camel, a mouse, a rat, an alpaca, a vicuna, a guanaco or a lama.

70. The method of claim 67, further comprising administering to the skin and/or scalp of a non-human animal a composition comprising
(i) a polynucleotide comprising
(a) a nucleic acid sequence as shown in SEQ ID NO: 1 or 3,
(b) a nucleic acid sequence encoding an amino acid sequence as shown in SEQ ID NO: 2 or 4,
(c) a nucleic acid sequence encoding an amino acid sequence as shown in SEQ ID NO: 2 or 4 having a modified signal peptide, a modified N- terminus and/or a modified C-terminus, or
(d) a nucleic acid sequence which hybridises under stringent conditions to any one of (a) to (c);
(ii) a polypeptide encoded by the nucleic acid as defined in any one of (a) to (c); or
(iii) a compound which binds to an antibody which specifically recognizes the polypeptide defined in (ii) or which specifically binds to an IL- 15 receptor alpha chain.

71. The method of claim 67, further comprising obtaining the hair of said non-human animal.

72. A method for manufacturing non-human animal hair comprising administering to the skin and/or scalp of a non-human animal the composition
(i) a polynucleotide comprising
(a) a nucleic acid sequence as shown in SEQ ID NO: I or 3,
(b) a nucleic acid sequence encoding an amino acid sequence as shown in SEQ ID NO: 2 or 4,
(c) a nucleic acid sequence encoding an amino acid sequence as shown in SEQ ID NO: 2 or 4 having a modified signal peptide, a modified N- terminus and/or a modified C-terminus, or
(d) a nucleic acid sequence which hybridises under stringent conditions to any one of (a) to (c);
(ii) a polypeptide encoded by the nucleic acid as defined in any one of (a) to (c); or
(iii) a compound which binds to an antibody which specifically recognizes the polypeptide defined in (ii) or which specifically binds to an IL-15 receptor alpha chain.

73. The method of claim 72, further comprising obtaining the hair of said non-human animal.

74. The method of claim 72, wherein said animal is a dog, a cat, a horse, a rabbit, a sheep, a camel, a mouse, a rat, an alpaca, a vicuna, a guanaco or a lama.


SEQUENCE LISTING (7 sequences)

SEQ ID NO: 1 - DNA, 1968 bp, Homo sapiens
actccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggaggtacagagctcggcttctttgccttgggaggggagtggtggtggttgaaagggcgatggaattttccccgaaagcctacgcccagggcccctcccagctccagcgttaccctccggtctatcctactggccgagctgccccgccttctcatggggaaaacttagccgcaacttcaatttttggtttttcctttaatgacacttctgaggctctcctagccatcctcccgcttccggaggagcgcagatcgcaggtccctttgcccctggcgtgcgactccctactgcgctgcgctcttacggcgttccaggctgctggctagcgcaaggcgggccgggcaccccgcgctccgctgggagggtgagggacgcgcgtctggcggccccagccaagctgcgggtttctgagaagacgctgtcccgcagccctgagggctgagttctgcacccagtcaagctcaggaaggccaagaaaagaatccattccaatatatggccatgtggctctttggagcaatgttccatcatgttccatgctgctgacgtcacatggagcacagaaatcaatgttagcagatagccagcccatacaagatcgtattgtattgtaggaggcatcgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgcctgtgtggaaccactgactactggctctcattgacttccttactaagcatagcaaacagaggaagaatttgttatcagtaagaaaaagaagaactatatgtgaatcctcttctttacactgtaatttagttattgatgtataaagcaactgttatgaaataaagaaattgcaataactggcaaaaaaaaaaaaaaaaaaaaaaaaa

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - DNA, 1312 bp, Mus musculus
ccacgcgtccgcaatactcagtggcactgtattccccttctgtccagccactcttccccagagttctcttcttcatcctcccccttgcagagtagggcagcttgcaggtcctcctgcaagtctctcccaattctctgcgcccaaaagacttgcagtgcatctccttacgcgctgcagggaccttgccagggcaggactgcccccgcccagttgcagagttggacgaagacgggatcctgctgtgtttggaaggctgagttccacatctaacagctcagagagaatccaccttgacacatggccctctggctcttcaaagcactgcctcttcatggtccttgctggtgaggtccttaagaacacagaaacccatgtcagcagataaccagcctacaggaggccaagaagagttctggatggatggcagctggaagcccatcgccatagccagctcatcttcaacattgaagctcttacctgggcattaagtaatgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctgactgcatgcgagcctcttccgtgtttctgttattaaggtacctccacctgctgctcagaggcagcacagctccatgcatttgaaatctgctgggcaaattaagcttcctaacaaggagataatgagccacttggatcacatgaaatcttggaaatgaagagaggaaaagagctcgtctcagacttatttttgcttgcttatttttaatttattgcttcatttgtacatatttgtaatataacagaagatgtggaataaagttgtatggatattttatcaattgaaaaaaaaaaaaaaaaaaaaaaaaaaaaaaaaaaaaaaaaaaaaaaaaaa

SEQ ID NO: 4 - protein, 162 aa, Mus musculus
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 5 - DNA, 25 bp, Artificial Sequence
  Synthetic Oligonucleotide
caatgatatacactgtttgagatga

SEQ ID NO: 6 - DNA, 23 bp, Artificial Sequence
  Synthetic Oligonucleotide
cgtgttgatgaacatttggacaa

SEQ ID NO: 7 - DNA, 1250 bp, Artificial Sequence
  Synthetic Construct
cttctgtccagccactcttccccagagttctcttcttcatcctcccccttgcagagtagggcagcttgcaggtcctcctgcaagtctctcccaattctctgcgcccaaaagacttgcagtgcatctccttacgcgctgcagggaccttgccagggcaggactgcccccgcccagttgcagagttggacgaagacgggatcctgctgtgtttggaaggctgagttccacatctaacagctcagagaggtcaggaaagaatccaccttgacacatggccctctggctcttcaaagcactgcctcttcatggtccttgctggtgaggtccttaagaacacagaaacccatgtcagcagataaccagcctacaggaggccaagaagagttctggatggatggcagctggaagcccatcgccatagccagctcatcttcaacattgaagctcttacctgggcattaagtaatgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctgactgcatgcgagcctcttccgtgtttctgttattaaggtacctccacctgctgctcagaggcagcacagctccatgcatttgaaatctgctgggcaaactaagcttcctaacaaggagataatgagccacttggatcacatgaaatcttggaaatgaagagaggaaaagagctcgtctcagacttatttttgcttgcttatttttaatttattgcttcatttgtacatatttgtaatataacagaagatgtggaataaagttgtatggatattttatcaattgaaatttaaaaaaaa